(19) United States
(12) Patent Application Publication
(10) Pub. No.: US 2005/0085661 A1
(43) Pub. Date: Apr. 21, 2005

(54) Esterfication process for production of fluoroalkyl (meth)acrylate by pervaporation-aided membrane reactor

(75) Inventors: Soo-Bok Lee, Seo-Gu (KR); Dong-Kwon Kim, Yuseong-Gu (KR); In Jun Park, Daedeok-Gu (KR); Jeong-Hoon Kim, Yuseong-Gu (KR); Kwang-Won Lee, Yuseong-Gu (KR); Jong-Wook Ha, Yuseong-Gu (KR); Kwang-Han Kim, Yuseong-Gu (KR); Sang-Man Ahn, Daedeok-Gu (KR); Yong-Taek Lee, Yuseong-Gu (KR)

Correspondence Address: FITZPATRICK CELLA HARPER & SCINTO, 30 ROCKEFELLER PLAZA, NEW YORK, NY, 10112, US

(73) Assignee: KOREA RESEARCH INSTITUTE OF CHEMICAL TECHNOLOGY, YUSUNG-GU (KR)

(21) Appl. No.: 10/882,185

(22) Filed: Jul. 2, 2004

(30) Foreign Application Priority Data
Oct. 17, 2003 (KR) 2003-72694

(51) Int. Cl. C07C 69/62

(52) U.S. Cl. 560/223

(57) ABSTRACT

The present invention relates to a process for preparing fluoroacrylic acid ester using a pervaporation composite membrane, in particular, to a process for preparing fluoroacrylic acid ester performed in such a manner that water and unreacted fluoroalcohol generated in esterification between fluoroalcohol and (meth)acrylic acid in the presence of an acid catalyst are condensed and then passed through a pervaporation membrane to effectively remove water, followed by recycling the unreacted fluoroalcohol removed of water. The present process exhibits much higher conversion rate of fluoroacrylic acid ester, allows the decrease of energy consumption and could be performed in environment-friendly manner.

### BACKGROUND OF THE INVENTION

1. Field of the Invention

The present invention relates to a process for preparing fluoroacrylic acid ester using a pervaporation composite membrane, in particular, to a process for preparing fluoroacrylic acid ester performed in such a manner that water and unreacted fluoroalcohol generated in esterification between fluoroalcohol and (meth)acrylic acid in the presence of an acid catalyst are condensed and then passed through a pervaporation membrane to effectively remove water, followed by recycling the unreacted fluoroalcohol removed of water. The present process exhibits an improved conversion rate of fluoroacrylic acid ester, allows for the decrease of energy consumption and could be performed in an environment-friendly manner.

2. Description of the Related Art

Esterified derivatives of fluoroacrylic acid exhibit excellent water-/oil-repelling properties, soil-repelling property and lower permittivity and surface energy, so that they have been used in a variety of fields such as optical fiber coatings, contact lens, and functional paints, solvent-typed thermoplastic paints, thermosetting paints, emulsion-typed paints and UV-typed paints with improved acid resistance, weather resistance, water repellency and oil repellency, water-/oil-repellents, soil repellents, and surface modifier coatings for paper, metal or polymer. Fluoroacrylic acid esters having such broader applications are expensive and estimated fifty thousand Wons(Korean currency)/kg. Their domestic demands have reached 60 bil. Wons in the year of 2001, and their worldwide market size is estimated about 500 billion Wons.

Fluoro(meth)acrylate, one of typical fluoroacrylic acid esters, can be prepared by esterification of fluoroalcohol and (meth)acrylic acid in the presence of an acid catalyst such as sulfuric acid or toluene sulfonic aicd according to Scheme 1.

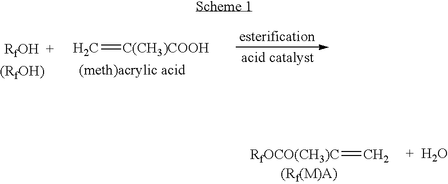

The above esterification is a reversible process and generates fluoro(meth)acrylate (R_{f}(M)A) as well as water (H₂O ) as by-products. To obtain the highest yield, it is necessary that the removal of water be rapidly performed for inhibiting the formation of thermodynamic equilibrium.

The raw material for producing fluoro(meth)acrylate, fluoroalcohol, is very expensive and impurities derived therefrom under the influence of fluorine cause to corrode a production apparatus. Therefore, various reactions are required to elevate the yield of the product and inhibit the production of water as a by-product. Fluorine has high electronegativity and small atom radius, so that it shows different reactivity from other atoms such as chlorine. Furthermore, the reaction involving the compounds with substituted fluorine can be hardly anticipated from the general knowledge of organic chemistry. Particularly, since fluoroalcohol involved in esterification has unique properties such as no hydrate formation with water and shows distinctly different reactivity from other alcohols, the reaction involving fluoroalcohol should be understood with the knowledge of fluorine chemistry.

Japanese Patent Publication Sho59-175452 discloses that fluoromethacylate (TFEMA) is synthesized by the esterification of fluoroalcohol (TFEA) and methacylic acid (MAA). Thereafter, using the reaction apparatus depicted in FIG. 1 embodying azeotropic distillation using solvents such as benzene, hexane and isopropylether, 5 volumes of n-hexane or the above solvents, are added to the product for extraction, and then washing and neutralizing are carried out with an equal volume of water and NaOH solution, respectively, finally yielding fluorinated TFEMA. However, since the process cannot promptly remove water produced during the reaction, its conversion rate becomes relatively low. In addition, for neutralizing conc. sulfuric acid as a reaction catalyst, a large amount of NaOH solution is required and the washing step should be repeatedly performed after the neutralization, so that a large amount of waste water is produced.

Japanese Patent Publication Sho59-181239 suggests that the product after esterification be fed out by vacuum distillation (40-160 mmHg), neutralized with NaOH solution and washed several times with water to give TFEMA. However, this method is also elucidated to have similar shortcomings to those of Japanese Patent Publication Sho59-175452 such as lower conversion rate and bulky production of waste water due to repeated washing with a large amount of water.

In this context, it could be recognized that the conventional esterification procedures for producing fluoroacrylate have some limitations as follows. Firstly, they consume a large amount of energy since the evaporation and condensation of organic solvents such as fluoroalcohol is repeatedly carried out before reaction. In particular, in the case of using fluoroalcohol with a lower boiling point, the unbalance of the equivalent ratio due to such evaporation becomes more prominent, and the consumption of high-cost fluoroalcohol during the step using a dehydrating agent exceeds 30%. Furthermore, the conventional esterification processes for producing fluoroacrylate are likely to contaminate surrounding environment owing to discharge or leakage of toxic fluorine-containing organic solvents, which is harmful to the health of workers and may also lead to explosion. To promote esterification during the process, the elevated temperature is required, which inevitably results in the production of polymerized materials such as dimers and by-products due to the action of an acid catalyst and high electronegativity of fluorine. Accordingly, to minimize the production of dimers and by-products, the process should be proceeded with extreme caution and additional steps for removing dimers and azeotropic solvents for dehydration of final products are required. In this regard, the production yield of high-cost monomers such fluorine-containing ester monomer and its quality are likely to be sharply dropped.

To solve the problems described above, those skilled in the art recently have focused on technologies relating to pervaporation composite membrane for selective separation of water.

The technology of pervaporation composite membrane had been developed in the late of 1980s by GFT Inc. in Germany. The composite membrane is prepared by coating porous support with water-soluble polymer such as polyvinylalcohol (PVA) in cross-linking manner. The composite membrane has a relatively high selectivity to small-sized water resulting from its higher affinity and elaborate structure. In particular, since PVA shows higher affinity to water, the membrane is mainly used to separate materials to give azeotropic point such as water-ethanol, water-isopropyl alcohol and water-acetic acid, which require high-energy consumption in conventional distillation processes. Since the separation using the composite membrane is operated at a temperature lower than distillation temperature, its energy consumption is expected to be ⅓-1/10 of that of conventional distillation, thereby allowing the separation using pervaporation composite membrane to largely replace the distillation process.

Recently, the researchers focusing on esterification in USA, Japan and Europe have made intensive studies on adopting the pervaporation composite membrane to esterification reactor for both reaction and separation with low energy consumption.

In the present invention, as approaches to be freed from the shortcomings of fluoroalcohol esterification described previously, the pervaporation composite membrane was applied to esterification procedures. That is, the present inventors have anticipated that, since the process using pervaporation composite membrane is operable at a lower temperature and closed type to selectively separate water, the process applied to fluoroalcohol esterification would allow to avoid the requirement of organic solvents and to highly elevate the conversion rate without the production of a by-product such as dimers in a more convenient manner.

### SUMMARY OF THE INVENTION

The present inventors have conducted extensive researches to overcome shortcomings of low conversion rate of fluoroacrylic acid ester due to water generated as a by-product in esterification between fluoroalcohol and (meth)acrylic acid and massive production of waste water. As a result, the present inventors have found that water can be eliminated more effectively if a condensate of unreacted fluoroalcohol and water generated as a by-product in the esterification are passed across a pervaporation membrane.

Accordingly, it is an object of this invention to provide a process for producing a fluoroacrylic acid ester by esterification between fluoroalcohol and (meth)acrylic acid in a more effective manner.

### BRIEF DESCRIPTION OF THE DRAWINGS

FIG. 1 schematically represents a conventional process using azeotropic distillation for preparing fluoroacrylic acid ester.

FIG. 2 schematically represents the present process using a pervaporation composite membrane for preparing fluoroacrylic acid ester.

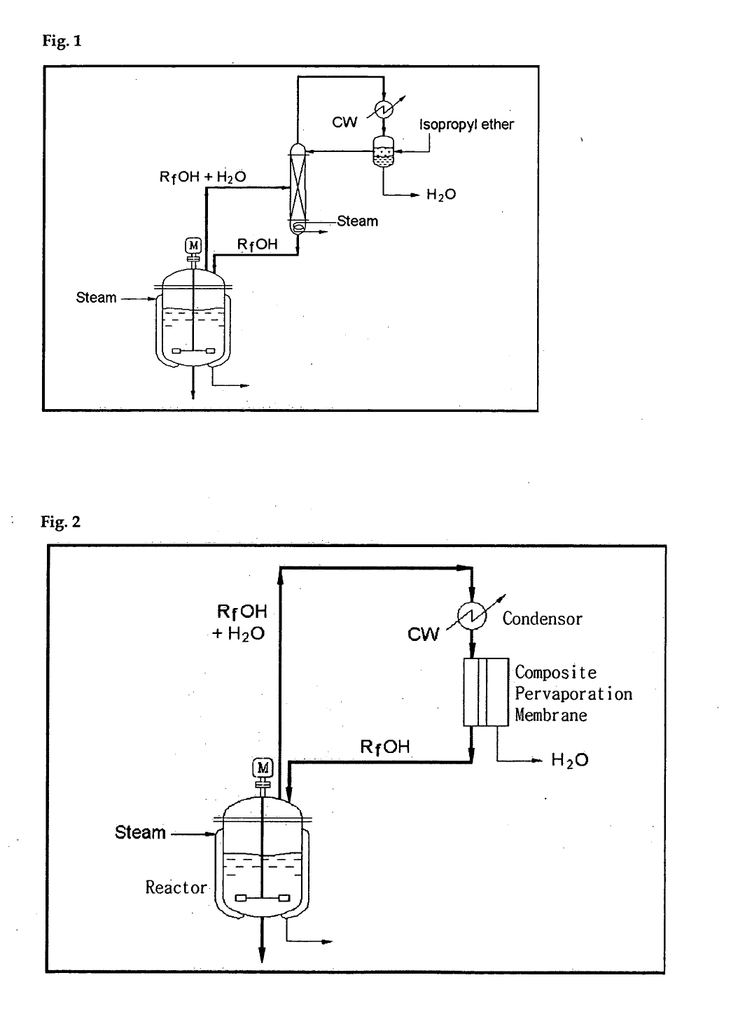

### DETAILED DESCRIPTION OF THIS INVENTION

In an aspect of this invention, there is provided a process for producing a fluoroacrylic acid ester by esterification between fluoroalcohol and (meth)acrylic acid in the presence of an acid catalyst, wherein the process further comprises condensing water generated as a by-product and unreacted fluoroalkyl alcohol, and passing the resultant through a pervaporation membrane to remove water.

The present invention will be described in more detail hereunder.

The present invention is directed to a process for producing a fluoroacrylic acid ester by esterification, in which to eliminate water as a by-product that highly affects the conversion rate to fluoroacrylic acid ester obtained by esterification of fluoroalcohol and (meth)acrylic acid, water and unreacted fluoroalcohol are condensed and then passed across a pervaporation membrane to effectively remove only water, followed by recycling the unreacted fluoroalcohol removed of water, thereby resulting in solving the problems in view of purity, yield, energy consumption and environment contaminant.

The striking technical feature of this invention lies in the employment of pervaporation membrane for removing water produced in esterification between fluoroalcohol and (meth)acrylic acid. Preferably, the pervaporation membrane has a selectivity ranging from 100 to 10000 in water/perfluoro alcohol mixed solution and water/acrylic acid mixed solution and a permeate flux ranging from 0.1-1 kg/m²·h. Within the ranges of selectivity and permeate flux, the present invention allows to recycle unreacted fluoroalcohol removed of water, so that it exhibits much higher the conversion rate of fluoroacrylic acid ester. If the selectivity is less than 100, perfluoroalcohol and acrylic acid are exposed to a permeation part. If the permeate flux is less than 0.1 kg/m²·h, the surface area of membrane is increased in parallel with treatment capacity, which results in the increase of cost for membrane. The pervaporation membrane to be used in this invention may include those used in the conventional processes, such as cross-linked polyvinyl alcohol or ethylene-vinylalcohol copolymer composite membrane, polysulfone, polyetherimide or polyacrylonitrile prepared by phase inversion process, and GFT membrane available from SchulzeChemtech, Inc.(Germany). The pervaporation membrane may be in any construct, including an asymmetric flat membrane, a tube or a hollow fiber membrane. The present invention permits to effectively separate water and unreacted fluoroalcohol and generate high-pure unreacted fluoroalcohol by use of pervaporation membrane, thereby drastically elevating the conversion rate of fluoroacrylic acid esters. The fluoroalcohol as a reactant of this invention is a compound represented by formula 1:
W(CF₂)l(X)ₘ(CH₂)ₙOH (1)
wherein W is —CF₃, —CF₂H, —CF₂Cl, —CF(CF₃)₂ or —CCl(CF₃)₂, X is —CH(OH), l and n are an integer of 0-20 and m is 0 or 1.

For example, the fluoroalcohol is at least one selected from the group consisting of CF₃CH₂OH, CF₃CF₂CH₂OH, CF₃CF₂CF₂CH₂CH₂OH, CF₃CCl(CF₃)(CF₂)₇CH₂OH, H(CF₂)₁₀CH₂OH, CF₂Cl(CF₂)₁₀CH₂OH, CF₃(CF₂)₇CH₂(OH)CHCH₂OH CF₃(CF₂)₄CH₂(OH)CHCH₂OH, CF₃(CF₂)₄CH₂OH, CF₃(CF₂)₆(CH₂)₂OH, CF₃(CF₂)₆CH₂OH, CF₃(CF₂)₇CH₂CH₂OH, (CF₃)₂CF(CF₂)₃CH₂OH, CF₃(CF₂)₇(CH₂)₄OH, (CF₃)₂CF(CF₂)₆(CH₂)₃OH, (CF₃)₂CF(CF₂)₆CH₂CH(OH)CH₂OH, CF₃(CF₂)₆(CH₂)₂OH and CF₃(CF₂)₈(CH₂)₂OH. It is demanded that the suitable fluoroalcohol is selected because the permeate flux of pervaporation membrane can be altered depending on the type of fluoroalcohol.

In addition, (meth)acrylic acid, which is used along with fluoroalcohol in the generation of fluoroacrylic acid esters by esterification, may include any compound containing acrylic acid, e.g., acrylic acid and methacrylic acid.

It is preferred that the fluoroalcohol is used in the amount of 1.5-3 moles relative to 1 mole of acrylic acid, thereby maintaining the mole ratio of [OH]/[H] in a reactor to 1-2 moles. If the mole ratio is less than 1, alcohol remained in the reactor is insufficient and therefore the active reaction cannot be anticipated; in the case of exceeding 3 moles, the energy consumption becomes greater due to excessive use of alcohol.

It is preferred that the acid catalyst for promoting esterification has a boiling point of no less than 200° . If the boiling point is less than 200° C., the catalyst is evaporated and therefore the chemical/mechanical stability of pervaporation composite membrane, which is labile to acid, becomes sharply dropped. For example, the acid catalyst is at least one selected from the group consisting of methanesulfonic acid, bezenesulfonic acid, toluenesulfonic acid, liquid/solid Nafion, phosphoric acid and a solid acid catalyst.

One specified embodiment of the present process for preparing a fluoroacrylic acid ester by esterification will be described as follows.

An acid catalyst, fluoroalkylalcohol and acrylic acid are introduced into a reactor(1) to perform esterfication. The reaction is carried out for 18-24 hr at a temperature ranging from 80 to 120° C. If the temperature is lower than 80° C., the forward reaction is retarded and therefore energy consumption becomes greater; in contrast, if it exceeds 120° C., acryl-typed dimers are generated or isomers resulting from esterification of fluoroalcohol are produced. A mixture of water as a by-product and unreacted fluoroalcohol is condensed in a condenser(2) to form liquid. The unreacted fluoroalcohol and water condensed are passed across the pervaporation composite membrane(3) and separated. The unreacted fluoroalcohol separated is recycled to be introduced to the reactor(1).

As described previously, the esterification between fluoroalcohol and acrylic acid according to the present invention makes it possible to produce fluoroacrylic acid ester in an environment-friendly manner with higher purity and yield.

The following specific examples are intended to be illustrative of the invention and should not be construed as limiting the scope of the invention as defined by appended claims.

### PREPARATION EXAMPLE

### Preparation of Pervaporation Composite Membrane

### Preparation Example 1

1% aqueous polyvinylalcohol (PVA) solution was mixed with 1% aqueous glutaraldehyde solution as a cross-linking agent and homogeneously agitated in the presence of a catalytic amount of acid catalyst. The resultant was coated on a polyimide porous support with a casting device to prepare PVA cross-linking composite membrane and thermally set at 120° C. to obtain a pervaporation composite membrane.

For applying the pervaporation composite membrane prepared to a membrane reactor, the experiments were carried out at 90° C. in 90/10 wt % of trifluoroethylalcohol/water solution, 95/5 wt % of water/acrylic acid solution or 99/1 wt % of water/trifluoroethylmethacrylate solution. As a result, for trifluoroethylalcohol/water, the selectivity was revealed as 150, 200 and 500 and the permeate flux of water as 0.5, 0.2 and 0.1 kg/m²/hr, respectively, which are evaluated as significantly high permeate flux. For acrylic acid/water, the selectivity was revealed as 1500, 2000 and 5000 and the permeate flux of water as 0.3, 0.2 and 0.1 kg/m²/hr, respectively, which are also evaluated as significantly high permeate flux. For the solution of water/trifluoroethylmethacrylate solution, the selectivity could not be evaluated due to water-immiscible property and for pure water/trifluoroethylmethacrylate solution, the permeate flux was not observed. Therefore, it suggests that the pervaporation composite membrane of Example 1 can be applied to a membrane reactor.

### Prepartion Example 2

For applying the GFT membrane available from SchulzeChemtech, Inc. (Germany) to a membrane reactor, the experiments were carried out at 90° C. in 90/10 wt % of trifluoroethylalcohol/water solution, 95/5 wt % of water/acrylic acid solution or 99/1 wt % of water/trifluoroethylmethacrylate solution.

As a result, for trifluoroethylalcohol/water, the selectivity was revealed as 130, 180 and 250 and the permeate flux of water as 0.4, 0.3 and 0.1 kg/m²/hr, respectively, which are evaluated as a significantly high permeate flux. For acrylic acid/water, the selectivity was revealed as 2000, 3000 and 4000 and the permeate flux of water as 0.25, 0.2 and 0.15 kg/m²/hr, respectively, which are also evaluated as significantly high permeate flux. For the solution of water/trifluoroethylmethacrylate solution, the selectivity could not be evaluated due to water-immiscible property and for pure water/trifluoroethylmethacrylate solution, the permeate flux was not observed. Therefore, it suggests that the pervaporation composite membrane of Example 2 can be applied to a membrane reactor.

### Prepartion Example 3

1% aqueous ethylene-vinylalcohol copolymer solution was mixed with 1% aqueous glutaraldehyde solution as a cross-linking agent and homogeneously agitated in the presence of toluene sulfonic acid catalyst. The resultant was coated on a polyimide porous support with a casting device to prepare a composite membrane and thermally set at 80° C. to obtain a pervaporation composite membrane.

The experiments were carried out at 90° C. in 90/10 wt % of perfluoroalcohol/water solution, 95/5 wt % of water/acrylic acid solution or 99/1 wt % of water/perfluoroalcohol methacrylate solution.

As a result, for trifluoroalcohol/water, the selectivity was revealed as 137, 154 and 140 and the permeate flux as 0.7, 0.4 and 0.2 kg/m²/hr, respectively, which are evaluated as a significantly high permeate flux. For methacrylic acid/water, the selectivity was revealed as 190, 250 and 350 and the permeate flux of water as 1.2, 0.5 and 0.2 kg/m²/hr, respectively, which are also evaluated as significantly high permeate flux. Therefore, it could be recognized that the pervaporation composite membrane formed at a relative—low temperature in Example 3 can be applied to a membrane reactor.

### Prepartion Example 4

The flat membrane was prepared using the solution containing 20 wt % of polysulfone, 30 wt % of acetone, 50 wt % of N-methylpyrrolidone and subject to evaporation for 1 min. Thereafter, the membrane was immersed in water to give an asymmetric membrane, followed by thermal treatment and washing to yield a pervaporation composite membrane.

The experiments were carried out at 90° C. in 90/10 wt % of trifluoroethylalcohol/water solution, 95/5 wt % of water/acrylic acid solution or 99/1 wt % of water/trifluoroethylmethacrylate solution.

As a result, for trifluoroethylalcohol/water, the selectivity was revealed as 237, 152 and 164 and the permeate flux as 1.7, 1.4 and 1.2 kg/m²/hr, respectively, which are evaluated as a significantly high permeate flux. For acrylic acid/water, the selectivity was revealed as 290, 350 and 340 and the permeate flux of water as 0.2, 0.7 and 0.3 kg/m²/hr, respectively. Therefore, it suggests that the pervaporation composite membrane derived from polysulfone asymmetric membrane of Example 2 can be applied to a membrane reactor.

### Preparation Example 5

The flat type asymmetric membrane was prepared using the solution containing 30 wt % of polyetherimide, 35 wt % of tetrahydrofuran, 30 wt % of dimethylformamide and subject to evaporation for 3 min. Thereafter, the membrane was immersed in water to give an asymmetric membrane, followed by thermal treatment and washing to yield a pervaporation composite membrane.

The experiments were carried out at 90° C. in 90/10 wt % of perfluoroalcohol/water solution, 95/5 wt % of water/acrylic acid solution or 99/1 wt % of water/ perfluoroalcohol methacrylate solution.

As a result, for trifluoroalcohol/water, the selectivity was revealed as 337, 252 and 264 and the permeate flux as 0.7, 0.4 and 0.2 kg/m²/hr, respectively, which are evaluated as significantly high permeate flux. For methacrylic acid/water, the selectivity was revealed as 390, 450 and 340 and the permeate flux of water as 0.32, 0.57 and 0.35 kg/m²/hr, respectively, which are also evaluated as significantly high permeate flux. Therefore, it could be recognized that the pervaporation composite membrane of polyetherimide asymmetric membrane of Example 5 can be applied to a membrane reactor.

### EXAMPLE

### Preparation of Fluoroacrylic acid ester Using Pervaporation Composite Membrane

### Example 1

78 g of methacrylic acid, 48 g of 2,2,2-trifluoroethanol, and 6 g of 95% toluene sulfonic acid (1.4-fold mole of alcohol) were introduced into a reactor(1) and mixed. The reaction was conducted in the reactor kept at 75° C. for 5 hr. Over the reaction, water produced along with unreacted fluoroalcohol was evaporated to discharge into an upper condenser(2) that generates liquid.

The unreacted fluoroalcohol and water as by-product condensed were passed to the pervaporation composite membrane(3) of PREPARATION EXAMPLE 1 to remove water and separate fluoroalcohol. The unreacted fluoroalcohol was recycled to be introduced to a reactor(1) and then reacted with methacrylic acid for conversion. The final reaction mixture was extracted with 5 volumes of n-hexane and water, washed with an equal volume of water and neutralized with 1N aqueous sodium hydroxide solution. The analysis was performed by gas chromatography, showing the conversion rate of 2,2,2-trifluoroethanol of 99%.

### Example 2

The same procedure as Example 1 was performed except that 77 g of methacrylic acid, 97 g of 2,2,3,3,3-pentafluoropropanol, 6 g of 95% Nafion and 10.0 g of phosphoric acid were used, and the reaction was conducted at 95° C. for 10 hr.

The final reaction mixture was extracted with 5 volumes of n-hexane and water, washed with an equal volume of water and neutralized with 1N aqueous sodium hydroxide solution. The analysis was performed by gas chromatography, showing the conversion rate of 2,2,2-trifluoroethylmethacylate of 98.7%.

### Example 3

The same procedure as Example 1 was performed except that the amount of methacrylic acid, 2,2,3,3,3-pentafluoropropanol and the solid acid catalyst was 2-fold, 1-fold and 5-fold, respectively, higher than those of Example 1. The reaction was performed using the polyimide pervaporation membrane for 5 hr at 110° C.

The final reaction mixture was extracted with 5 volumes of n-hexane and water, washed with an equal volume of water and neutralized with 1N aqueous sodium hydroxide solution. The analysis was performed by gas chromatography, showing the conversion rate of 2,2,2-trifluoroethylmethacylate of 99%.

### Comparative Example 1

The same procedure as Example 1 was performed except that 700 g of methacrylic acid, 25 g of 2,2,2-trifluoroethanol and 24 g of sulfuric acid were reacted for 8 hr at 135° C. A polyetherimide pervaporation membrane was used.

The reaction procedure was shown unsatisfactory because the pervaporation membrane was decomposed during the reaction.

### Comparative Example 2

The same procedure as Example 1 was performed except that 700 g of methacrylic acid, 25 g of 2,2,2-trifluoroethanol and 24 g of the solid acid catalyst were used and the distillation method was employed.

The final reaction mixture was extracted with 5 volumes of n-hexane and water, washed with an equal volume of water and neutralized with 1N aqueous sodium hydroxide solution. The analysis was performed by gas chromatography, showing the conversion rate of 2,2,2-trifluoroethylmethacylate of 49%. In addition, a large amount of impurities such as dimers were generated.

### Comparative Example 3

The same procedure as Example 1 was performed except that the conventional azeotropic distillation process was employed instead of pervaporation membrane. The final reaction mixture was extracted with 5 volumes of n-hexane and water, washed with an equal volume of water and neutralized with 1N aqueous sodium hydroxide solution. The analysis was performed by gas chromatography, showing the conversion rate of 2,2,2-trifluoroethylmethacylate of 70%.

Examples 1-3 using a pervaporation membrane of PREPARATION EXAMPLEs 1-5 according to the present invention exhibits much higher conversion rate compared to Comparative Example 3 using azeotropic distillation according to the conventional process. In addition, Comparative Examples 2 and 3 in which the reaction temperature is beyond the preferable range, respectively, resulted in membrane damage or a significantly low conversion rate.

### Examples 4-7

The reactions were carried out in the same manner as Example 1 using a pervaporation membrane prepared in Preparation Examples 2-5. The conversion rates of fluoroacrylic acid esters produced are shown in Table 1. As understood from Table 1, the present invention using a pervaporation membrane showed the conversion rate of no less than 98%, which is interpreted as being significantly effective.

TABLE 1

| Category | Pervaporation membrane | Conversion rate (%) |
|---|---|---|
| Ex. 1 | Prep. Ex. 1 | 99 |
| Ex. 4 | Prep. Ex. 2 | 98.7 |
| Ex. 5 | Prep. Ex. 3 | 99 |
| Ex. 6 | Prep. Ex. 4 | 98.5 |
| Ex. 7 | Prep. Ex. 5 | 98.3 |

As discussed previously, the present process in which the by-product is dehydrated through a pervaporation membrane and an unreacted reactant is recycled, exhibits much higher conversion rate of industrial-useful fluoroacrylic acid ester and saves energy by more than 40% compared to the conventional process. In addition, the present process is performed in environment-friendly manner and applicable to various compounds such as ethers and ketones.

1. A process for producing a fluoroacrylic acid ester by esterification between fluoroalcohol and (meth)acrylic acid in the presence of an acid catalyst, wherein said process further comprises:
condensing water, generated as a by-product, and unreacted fluoroalkyl alcohol; and
passing the resultant through a pervaporation membrane to remove water.

2. The process according to claim 1, wherein said pervaporation membrane has a selectivity ranging from 100 to 10000 in a water/perfluoro alcohol mixture and a water/acrylic acid mixture, respectively, and permeate flux ranging from 0.1-1 kg/m²·h.

3. The process according to claim 1, wherein a raw material of said pervaporation membrane is selected from the group consisting of a composite membrane of cross-linked polyvinyl alcohol or ethylene-vinylalcohol copolymer onto polyacrylonitrile, polysulfone, or polyetherimide support membrane prepared by phase inversion process.

4. The process according to claim 1 or 3, wherein said pervaporation membrane has an asymmetric flat membrane, a tube or a hollow fiber membrane.

5. The process according to claim 1 or 3, wherein said fluoroalcohol is a compound represented by formula 1:
W(CF₂)l(X)ₘ(CH₂)ₙOH (1)
wherein W is —CF₃, —CF₂H, —CF₂Cl, —CF(CF₃)₂ or —CCl(CF₃)₂, X is —CH(OH), l and n is an integer of 0-20, respectively, and m is 0 or 1.

6. The process according to claim 1 or 3, wherein said fluoroalcohol is selected from the group consisting of CF₃CH₂OH, CF₃CF₂CH₂OH, CF₃CF₂CF₂CH₂CH₂OH, CF₃CCl(CF₃)(CF₂)₇CH₂OH, H(CF₂)₁₀CH₂OH, CF₂Cl(CF₂)₁₀CH₂OH, CF₃(CF₂)₇CH₂(OH)CHCH₂OH, CF₃(CF₂)₄CH₂(OH)CHCH₂OH, CF₃(CF₂)₄CH₂OH, CF₃(CF₂)₆(CH₂)₂OH, CF₃(CF₂)₆CH₂OH, CF₃(CF₂)₇CH₂CH₂OH, (CF₃)₂CF(CF₂)₃CH₂OH, CF₃(CF₂)₇(CH₂)₄OH, (CF₃)₂CF(CF₂)₆(CH₂)₃OH, (CF₃)₂CF(CF₂)₆CH₂CH(OH)CH₂OH, CF₃(CF₂)₆(CH₂)₂OH and CF₃(CF₂)₈(CH₂)₂OH.

7. The process according to claim 1, wherein said acid catalyst is at least one selected from the group consisting of methanesulfonic acid, bezenesulfonic acid, toluenesulfonic acid, liquid/solid Nafion, phosphoric acid and solid acid catalyst; in which said acid catalyst has a boiling point of no less than 200° C.

8. The process according to claim 1 or 3, wherein said esterification is performed for 18-24 hr at a temperature ranging from 80 to 120° C.
